(19) United States
(12) Patent Application Publication
(10) Pub. No.: US 2003/0228383 A1
(43) Pub. Date: Dec. 11, 2003

(54) Herbal cough formulations and process for the preparation thereof

(75) Inventors: Madhukat Mansukhlal Doshi, Maharashtra (IN); Shashikant Avantilal Vasavada, Gujarat (IN); Milind Dattatraya Joshi, Maharashtra (IN); Shirish Bhagwanlal Mody, Maharashtra (IN)

Correspondence Address: LACKENBACH SIEGEL, ONE CHASE ROAD, SCARSDALE, NY, 10583, US

(73) Assignee: J.B. Chemicals & Pharmaceuticals, Ltd., Bombay (IN)

(21) Appl. No.: 10/163,948

(22) Filed: Jun. 6, 2002

(51) Int. Cl. A61K 35/78

(52) U.S. Cl. 424/734; 424/756; 424/757; 424/777

(57) ABSTRACT

The present invention relates to herbal composition for the treatment of chronic respiratory disorders such as cold, cough, allergic asthma, seasonal allergic rhinitis, pharyngitis, laryngitis and the like and a process for preparing the same. The composition comprises extracts derived from Ayurvedic plants selected from the following group:

| Botanical name | Quantity in % |
|---|---|
| Ocimum sanctum | 10-40% |
| Glycerrhiza glabra | 10-25% |
| Curcuma longa | 2-20% |
| Zingiber officinale | 1-10% |
| Adhatoda vasika | 10-25% |
| Solanum indicum | 1-10% |
| Saussurea lappa | 1-5% |
| Piper cubeba | 1-5% |
| Terminalia belerica | 1-10% |
| Aloe barbadensis | 10-30% |
| Inula racemosa | 2-10% |
| Clerodendrum serratum | 1-5% |
| Solanum xanthocorpum | 1-5% |
| Pipper longum | 1-5% |
| Alpinia galanga | 1-5% |
| Terminalia chebula | 5-15% |
| Embelica officinalis | 5-15% |

The process for preparing the herbal composition of the present invention comprises procuring, cleaning, grading of specified herbal plants as per the standard specification, disintegrating and pulverizing separately to form the coarse powder, then macerating individual powders separately with aqueous solution of preservatives and then extracting, filtering, concentrating and spray drying to make dry extract powder or semisolid soft extract. Further dissolving each of the extracts separately in aqueous solution containing preservatives and then allowing to maturate after mixing. Then adding filtered extracts to the sugar syrup containing preservatives, glycerin and appropriate excipients to obtain a homogenous cough syrup. The herbal composition of the present invention is non alcoholic, non sedating and non-freezing in nature and the process of preparation as described above does not employ pharmaceutically unacceptable organic solvents.

### TECHNICAL FIELD

The present invention relates to herbal formulation and a method for preparation thereof for providing treatment in chronic respiratory disorders such as cold, cough, allergic asthma, seasonal allergic rhinitis, pharyngitis, laryngitis, whooping cough and the like and which is non alcoholic, non sedating and non-freezing in nature.

### BACKGROUND OF INVENTION

During the last two decades there has been an increasing interest in utilization of Indian Medicinal plants for treating chronic respiratory disorders as some of these plants are known to be used in traditional medicine for at least alleviating the symptoms associated with them.

Ayurvedic System of Medicine originated in India more than 5000 years ago, utilizes the curative properties of plants for treating diseases. Ayurveda recognizes three different humors, Vata, Pitta and Kapha, which control the body metabolism. Vata controls all the movements of the body as it releases energy mainly from the catabolic reactions, while Pitta is responsible for the enzymatic reactions responsible for both anabolic and catabolic pathways. However, Kapha is responsible for anabolic reactions, which are responsible for all synthetic compounds deposited in the body. A harmonious working of the above three humors in a balanced manner is responsible for the optimum functioning of a healthy individual. Any imbalance in the harmonious working of the three humors leads to imbalance of body metabolism as well as body functions, which ultimately result, into different ailments. The primary purpose of Ayurvedic System of Medicine is to bring about a balanced state of three humors and to exert prophylactic or curative effects.

Phytochemical investigation of the herbal drugs or their extracts reveals the presence of several chemical compounds. Though all the ingredients do not possess specific therapeutic activity, it is believed that the different chemical compounds present in the plant or its extract act in harmonious manner and possibly due to synergistic effect of the compounds, some ingredients potentiate the therapeutic activity of the main active ingredient or even retard the undesirable actions of the main compound because individual compounds may not be as effective as that of the mixture of compounds present in the plant.

Multi-ingredient therapy is preferred over single drug therapy since the therapy may be effective in any one of these three humors i.e. Vata, Pitta & Kapha for the manifestation of the diseases. At the same time, most of the diseases are associated with imbalance of more than one humor producing different symptoms involving various organs of the body. Hence a multi-ingredient therapy includes a combination of herbal drugs which may have either a complimentary or supplementary action for each other.

Based on these principles of Ayurveda, the inventors have developed a herbal cough remedy formulation with selected herbal ingredients that bring about a balance of three basic humors present in the body thereby providing improved treatment by administering a safe and effective amount of composition comprising of such ingredients that treats in all types of coughs.

There are many patents related to innovative herbal products but employed mainly for the treatment of skin disorders, fungal infections, arthritic and joint pain, various inflammatory conditions, psoriasis, dementia and few CNS disorders. There are very few patents which describe compositions for treatment of respiratory disorders.

U.S. Pat. No. 6,193,995 describes herbal mixture for the treatment of nasal congestion that comprises of plurality of herbs thoroughly mixed together and combined with an adhesive. This is a type of herbal bandage made of a mixture of white pepper, dried ginger rhizome and cinnamon twig powder with adhesive. The present invention differs from the above patent because the patented abstract is a topical dosage form and it is for the treatment of nasal congestion whereas present invention is an oral syrup formulation for treating cough from different etiologies and related symptoms.

U.S. Pat. No. 6,287,567 describes a method of making an herbal drink and an apparatus developed for relieving symptoms of fatigue, congestion, fever and asthma. The drink is formed by straining water through a combination of equal portions of rosehip, goldenseal, comfrey leaf, bee pollen, spearmint, chickweed, comfrey root, chamomile flower, catnip, mullein, pennyroyal, eucalyptus, and licorice root. The therapy does not address severe conditions such as bronchial asthama, chronic obstructive pulmonary disease, bronchitis and other respiratory disorders and also the product is not cost-effective since it involves special apparatus for administration of the medicament.

U.S. Pat. No. 6,197,306 describes herbal composition mainly for the treatment of chronic obstructive pulmonary disease (COPD) and respiratory disorders and a process for preparing the same. The composition comprises of Bryonia, Ipecacuanha, Drosera and Antimony potassium tartrate. The product preparation involves use of pharmaceutically hazardous organic solvents for extraction process.

There is a need to provide a herbal composition having an improved therapeutic efficacy for the treatment of various types of cough and respiratory disorders.

In view of the foregoing shortcomings inherent in the known types of herbal compositions as mentioned in the prior art, the present invention provides a novel herbal composition wherein the same can be utilized for relieving cough from different etiology and also related symptoms.

There is a further object of the present invention to provide a process for preparation of herbal composition, which is devoid of use of pharmaceutically unacceptable organic solvents.

It is still further object of the present invention to provide herbal composition which does not contain codeine or any other sedative agent which causes side effects like dizziness, delirium, etc.

It is still further object of the present invention to provide herbal composition, which does not freeze, at subzero temperatures thus preserving the medicinal properties of the herbs and thereby making it more efficacious and better patient compliant.

It is still further object of the present invention to provide herbal composition which does not contain self generated or externally added alcohol thus preventing abuse generally associated with common cough remedies.

To attain this, the composition of present invention is developed which comprises of herbal mixture of following plants for the preparation of the composition of the present invention.

Ocimum sanctum

Therapeutically used parts are leaves, seeds and roots. The leaves contain essential oil, which exert anti-microbial activity against M. tuberculosis and S. aureus and other bacteria and fungi. A tea prepared from leaves is commonly used in cough, cold, mild indigestion, diminished appetite and malaise The solid extract significantly relieves the breathlessness with tropical eosinophilia. It is commonly used in bronchial asthma. The seeds are used as a general tonic.

Glycerrhiza glabra

Therapeutically used parts are peeled roots. It is extensively used extensively as a demulcent, mild expectorant and anti-inflammatory agent. Anti-inflammatory action is attributed to glycerrhetinic acid. It relieves vata and kapha inflammations, it is also used in eye diseases, throat infections, symptomatic relief in peptic ulcer and as an antiarthritic agent.

Zingeber officinale

Therapeutically used parts are scrapped, dried as well as green roots. The active principles gingerol, dehydrogingerdione, gingerdione exhibit anti-inflammatory activity. Antihistaminic activity has also been shown. It is indicated against Nausea, anorexia, dyspepsia, motion sickness, common cold.

Curcuma longa

Therapeutically used parts are tubers and rhizomes. Aqueous extracts of turmeric showed significant anti-inflammatory activity. Stabilizing report on lysosomal membranes are also reported. Application of powder on septic as well as aseptic wounds accelerated the process of healing to the extent of 23-24%. It is indicated in the treatment of sore throat, wounds, urticaria and skin allergies.

Adhatoda vasica

Therapeutically used parts are leaves, roots, flowers and bark. The leaves of the plant contain essential oil and alkaloid vasicine, N-oxides of vasicin, vasicinone, deoxyvasicine and maiontone. The roots are known to contain vasicinolone, vasicol, peganine and 2-hydroxy-4-glucosyl-oxychalcone. The flowers contain β-sitosterol-D-glucoside, kaempferol, its glycosides and quercetin. The leaf extract has been used for treatment of bronchitis and asthama for many centuries. It relieves cough and breathlessness. Large doses of fresh juice of leaves have been used in tuberculosis.

Solanum indicum

Therapeutically used parts are fruits, seeds and roots. It is described as useful in asthma, dry cough, chronic febrile afflictions and also in dysuria. The fruit is useful in leucoderma, pruritis and bronchitis.

Inula recemosa

Therapeutically used parts are roots. The plant extract showed potent anti-inflammatory, anti-pyretic and antispasmodic effect against bronchial spasm induced by histamin, 5-hydroxytryptamine and various plant pollens. The essential oil is moderately effective against some bacteria such as S. aureus. Ps. Aeruginosa, B. subtilis, E. coli, B. anthacis and fungus. In Ayurvedic practice, it is mainly used as an expectorant and brochodialator.

Piper cubeba

Therapeutically used parts are dried immature full grown fruits called the cubebs. The activity of the drug is attributed to the volatile oil, natural resin and cubebic acid.

Saussurea lappa

Therapeutically used parts are roots. It is used as a bronchodialator and antispasmodic, and also in skin diseases. The alkaloid saussarine has a smooth muscle relaxant activity. The essential oil has antiprotozoal and anti-bacterial effect.

Clerodendrum serratum

Therapeutically used parts are leaves and roots. A decoction of roots is used in asthma and bronchitis. It is also used in fever and also used in sinusitis.

Solanum xanthocarpum

Therapeutically used parts are leaves, flowers, fruits, stem and roots. It is an expectorant given in dosage of 20-30 g of the crude drug as decoction. Preliminary clinical trials have shown significant improvement in some respiratory diseases like bronchial asthma. Drug has shown antipyretic activity and its fatty acid oil possesses significant antifungal activity.

Piper longum

Therapeutically used parts are immature berries and roots. A common use of fruit is in the prevention of recurrent attacks of bronchial asthma.

Alpinia galanga

Therapeutically used parts are fruits and rhizomes. Essential oil from rhizomes was found active against gram positive and gram negative microorganisms. The rhizomes are useful in rheumatism and catarrehal infections. The volatile oil is used in cough, respiratory troubles and digestive mixtures.

Terminalia belerica

Therapeutically used parts are fruits which contain green fixed oil, saponin, tannins, rasinous residue and three amorphous and hygroscopic glycosidal compounds. Alcoholic extract of fruit shows marked bile stimulant activity. The clinical trial indicated that drug has bronchodialatory, antispasmodic, anti-phlegmatic, expectorant and sedative activities.

Aloe barbadensis

Therapeutically used parts are leaves, juice and pulp. Three main constituents of the plant are barbaloin, isobarbaloin and β-barbaloin. The plant is used as appetite stimulant, tonic in small doses and in larger doses as purgative. The leaf extract along with sugar is used in coughs and cold. The Ayurvedic preparation kumari Asava is used in several ailments such as general debility, cough, dyspnoea, asthama etc. It is also used in jaundice.

Embelica officinalis

Drug consists of fresh and dried fruits of Embelica officinalis. It is effective in treatment of peptic ulcer and in dyspepsia. The extract has antimicrobial properties. It is an antioxidant with free radical scavenging properties which may be due to the presence of high levels of super oxide dimutase.

Terminalia chebula

Therapeutically used part is fruit. Fruits contain about 30% of astringent substances such as chebulinic acid, tannic acid, gallic acid etc. Resin and a purgative priciples of the nature of anthraquinone and sennoside are also present. The extracts of the powdered fruits exhibit a wide anti-bacterial and antifungal spectrum. The friut pulp also exhibits laxative activity. It is used extensively in the preparations of many Ayurvedic formulations for infectious diseases which impart immunity and body resistance against diseases and also have beneficial effects on all the tissues.

### SUMMARY OF THE INVENTION

The present invention is based on the discovery that an herbal composition comprising of specific herbs properly extracted and blended in correct proportions will safely and significantly treat coughs from different etiology.

Allopathic cough syrups are commercially marketed from a long time and contain ingredients with one of the properties such as cough suppressants, decongestants, antihistaminic, antitussives and expectorants. These are associated with various side effects.

All the known cough syrups are effective in a particular type of cough only, e.g. some are effective against common cough and cold or others are effective against bronchitis or asthma, few against pneumonia and the like. The cough syrup which is now developed by the inventors has not been known and prepared by blending precise amounts of specially selected herbs for the treatments of coughs associated with various respiratory disorders.

The present herbal formulation is developed so as to provide relief in all types of chronic respiratory disorders such as cough, cold, allergic asthma, seasonal allergic rhinitis, pharyngitis, laryngitis, whooping cough and the like. It is for the first time that such a formulation is developed comprising of such ingredients, which helps in balancing of all types of coughs.

It is a known fact that most of the Ayurvedic cough syrups have self generated alcohol as these cough syrups are prepared from plants which generate alcohol on its own, or sometimes alcoholic extracts of the herbs are employed during preparation of such compositions. The present composition of the invention neither generates alcohol on its own nor is added externally. Thus, cough syrup of the present invention is alcohol free, thus avoiding the abuse associated with alcoholic compositions.

It is unlike other cough syrups, as it does not contain codeine or any other sedative agent. Codeine present in the known cough syrups causes major side effects such as drug abuse and constipation.

The present herbal formulation is developed in such a manner that the precise blend of specified herbal extracts and selected excipients, is non-freezing at subzero temperature thus preserves therapeutic efficacy of the herbal constituents at freezing temperatures without changing the state of formulation and hence provides better efficacy. This ensures delivering the composition with maximum efficacy for treatment of all types of coughs associated with respiratory tract diseases such as cold/allergic asthma, seasonal allergic rhinitis, pharyngitis, laryngitis, whooping cough and the like, at various parts of the world where subzero temperature is prevailing. The respiratory disorders are prevalent in cold seasons. The temperature goes below zero degrees in most of the parts of the world causing liquids to freeze and imparting inconvenience during administration. The nonfreezing nature of the composition of the present invention leads to improved patient compliance as it can be readily self administered by patient as and when required.

Also the presence of Aloe barbadensis as one of the active ingredients in the present—composition imparts an inherent mild desirable laxative activity. Furthermore, administering such a cough preparation leads to mucous liquidation process wherein excess mucous is excreted along with the faeces thereby supplementing laxative activity of Aloe barbadensis. Saponins from Terminalia belerica with surfactant action responsible for detaching sticky cough from bronchioles. They also act as mucolytics.

Thus, the present invention has taken care of all the untoward effects accompanied by the cough syrups already known and exhibits better efficacy for the treatment of cough associated with various types of respiratory disorders.

The basis of preparation of novel herbal formulation comprises of various therapeutically active Ayurvedic medicinal plants having the following therapeutic properties as given below—to be selected from the following medicinal plants:—

| Botanical name of Plant | Therapeutic Properties |
|---|---|
| Ocimum sanctum | has an anti-inflammatory antibacterial and |
| | stimulating expectorant action. |
| Glycerrhiza glabra | has an anti-inflammatory, demulcent and |
| | expectorant action. |
| Zingiber officinale | has an anti-inflammatory, spasmolytic action |
| | and anti-bacterial action. |
| Curcuma longa | has an analgesic, anti-inflammatory, and anti- |
| | bacterial action. |
| Adhatoda vasika | has an expectorative, spasmolytic, broncho- |
| | dialatory action. |
| Solanum indicum | has an antipyretic action, |
| Inula racemosa | has an anti-inflammatory, expectorative, |
| | spasmolytic and antitussive action. |
| Terminalia belerica | has an anti-oedematic, expectorative and |
| | antibacterial action. |
| Aloe barbadensis | has an antibacterial action. |
| Saussurea lappa | has a bronchodialator action. |
| Clerodendron serratum | has an anti-inflammatory, expectorative and |
| | antihistaminic action. |
| Solanum xanthocarpum | has an aperient, digestive, and alterative action |
| Piper longum | has an antitubercular action. |
| Alpinia galanga | has an antibacterial, spasmolytic action. |
| Piper cubeba | has an antiseptic and expectorant action. |
| Terminalia chebula | has a antibacterial and antifungal spectrum. |
| Embelica officinalis | has a antimicrobial and antiulcer properties |

### DETAILS OF THE INVENTION

The present invention relates to a herbal formulation containing a mixture of specified herbal ingredients and a method for preparation thereof for providing treatment in chronic respiratory disorders such as cold, cough, allergic asthma, seasonal allergic rhinitis, pharyngitis, laryngitis whooping cough and the like and which is non alcoholic, non sedating and non-freezing in nature. Further the process for the preparation of herbal composition of the present invention does not involve use of pharmaceutically unacceptable and hazardous organic solvents.

The process for preparing the herbal composition of the present invention comprises procuring, cleaning, grading of specified herbal plants as per the standard specification, disintegrating and pulverizing separately to form the coarse powder, then macerating individual powders separately in aqueous solution of preservatives and then extracting, filtering and concentrating and spray drying to make dry extract powder or semisolid soft extract. Further dissolving each of the extracts separately in aqueous solution containing preservatives and then allowing to maturate after mixing. Then adding filtered extracts to the sugar syrup containing preservatives, glycerin and appropriate excipients to obtain a homogenous cough syrup. The herbal composition of the present invention is non alcoholic, non sedating and non-freezing in nature and the process of preparation as described above does not employ pharmaceutically unacceptable organic solvents.

The process of manufacturing of herbal extracts is as follows:

1. The requisite quantities of the specified, cleaned, graded and dry powdered herbal ingredients are extracted by the conventional process at a temperature not exceeding 70° to 80° C.

2. Each of the extract is filtered separately, concentrated under vacuum at a temperature ranging between 50-55° C.

3. The concentrated extract is spray dried to dry powder or semisolid soft extract, which are used in the manufacturing process.

The product as set out in this specification is produced by a process as herein described, is advantageous as it ensures quicker and more efficient extraction of active ingredients at relatively low temperature (about 70° to 80° C.) without their undergoing any change/degradation.

This new herbal remedy formulation of plant ingredients are claimed by this specification can be prepared by the improved process comprising of steps as set out below:

Step I

Procuring of following herbal plants as per the standard specification.

For the purpose of this invention the Ayurvedic plants constituents are selected from the following group:

| Botanical name of | | Qty. in |
|---|---|---|
| Plant | Plant constituents | percentage. |
| Ocimum sanctum | leaves, seeds and root. | 10-40% |
| Glycerrhiza glabra | peeled root, | 10-25% |
| Zingiber officinale | scraped and dried rhizomes as | 1-10% |
| | well as the green ones. | |
| Curcuma longa | Tubers and rhizomes. | 2-20% |
| Adhatoda vasika | Leaves, roots, flowers and bark. | 10-25% |
| Solanum indicum | Roots, fruits and seeds. | 1-10% |
| Inula racemosa | Roots | 2-10% |
| Piper cubeba | Dried immature full grown fruits | 1-5% |
| | called the cubebs. | |
| Terminalia belerica | Fruits. | 1-10% |
| Aloe barbadensis | Expressed and dried juice of | 10-30% |
| | leaves and pulp. | |
| Saussurea Lappa | Roots. | 1-5% |
| Clerodendron serratum | Roots, Leaves. | 1-5% |
| Solanum xanthocorpum | Roots, stem, leaves, flowers | 1-5% |
| | fruits (panchang). | |
| Piper longum | Immature berries (i.e. dried, | 1-10% |
| | 1-5% unripe fruits or fruiting | |
| | spikes, dried in the sun and | |
| | roots. | |
| Alpinia galanga | Rhizome, fruit | 1-5% |
| Terminalia chebula | Fruits | 5-15% |
| Embelica officinalis | Fresh and dried fruits. | 5-15% |

Step II: Disintegrating each of the said herbal plants as given in Step I in the disintegrater in a conventional manner separately and pulverizing the same in the pulverizer to form the coarse powder.

Step-III: The above herbs are macerated separately by soaking the same in aqueous solution of preservatives such as sodium benzoate, sodium methylparaben, sodium propylparaben, sorbic acid, and then extracting it for 18 hours at 70°-80° C., which is then filtered, concentrated under vacuum and spray dried to make dry extract powder or semisolid soft extract.

Step IV:

The requisite quantities of each of the dry extract are again dissolved separately in aqueous solution containing requisite quantities of preservatives and then mixed together. The resulting solution is kept for maturation at 20-25° C. for 18 hrs and filtered.

Step-V:

Sugar syrup is prepared by dissolving in aqueous solution appropriate quantities of sugar, citric acid, preservatives, glycerin to obtain clear homogenous syrup. The filtered solution of extracts is added to syrup and stirred to obtain a homogenous syrup.

Step VI.

Colors, menthol and other flavouring agent along with other commonly known pharmaceutical excipients may be added as required to make this syrup palatable for patients' acceptabiliy.

The invention will be more fully understood from the following examples. These examples are to be constructed as illustrative of the invention and not limitative thereof:

### EXAMPLES

### Example I

The product has been formulated in the liquid dosage form having the following composition of active ingredients:

Composition of the Active Ingredients:

Each 10 ml contains extracts derived from:

| Name of Plant | Botanical name | Qty in % |
|---|---|---|
| Tulsi | Ocimum sanctum | 25% |
| Yastimadhu | Glycerrhiza glabra | 15% |
| Haridra | Curcuma longa | 12.5% |
| Sunthi | Zingiber officinale | 2.5% |
| Vasaka | Adhatoda vasika | 15% |
| Brahati | Solanum indicum | 5% |
| Pushkarmool | Inula racemosa | 5% |
| Sungadha muricha | Piper cubeba | 2.5% |
| Vibhitak | Terminalia belerica | 5% |
| Ghritakumari | Aloe barbadensis | 12.5% |

The specified ingredients are cleaned, graded, disintegrated and pulverized separately to form the coarse powder, then individual powders are macerated separately in aqueous solution of preservatives and then extracted, filtered, concentrated and spray dried to make dry powder extract. Further each of the extracts is dissolved separately in aqueous solution containing preservatives and then allowed to maturate after mixing. Then filtered extracts are added to the sugar syrup containing preservatives, citric acid, glycerin and appropriate excipients to obtain a homogenous cough syrup. The said composition is so adjusted as to deliver 400 mg of the total extracts in 10 ml of the liquid formulation.

### Example 2

The composition employed was similar to that in Example 1, except that the spray dried plant extracts used are semisolid in nature i.e. soft extract.

### Example 3

The product has been formulated in the liquid dosage form using similar manufacturing procedure as mentioned in example 1 and having the following composition of active ingredients:

Composition of the Active Ingredients:

Each 10 ml contains extracts derived from:

| Name of Plant | Botanical name | Qty in % |
|---|---|---|
| Tulsi | Ocimum sanctum | 21.27% |
| Yastimadhu | Glycerrhiza glabra | 12.67% |
| Haridra | Curcuma longa | 12.67% |
| Sunthi | Zingiber officinale | 3.19% |
| Vasaka | Adhatoda vasika | 17% |
| Bharangi | Clerodendron serratum | 8.5% |
| Kantakari | Solanum xanthocorpum | 4.25% |
| Pippali | Piper longum | 4.25% |
| Vibhitak | Terminalia belerica | 5.32% |
| Ghritakumari | Aloe barbadensis | 10.64% |

The said composition is so adjusted as to deliver 400 mg of the total extracts in 10 ml of the liquid formulation.

### Example 4

The composition employed was similar to that in Example 3, except that the spray dried plant extracts used are semisolid in nature i.e. soft extract.

### Example 5

The product has been formulated in the liquid dosage form using similar manufacturing procedure as mentioned in example 1 and having the following composition of active ingredients:

Composition of the Active Ingredients:

Each 10 ml contains extracts derived from:

| Name of Plant | Botanical name | Qty in % |
|---|---|---|
| Tulsi | Ocimum sanctum | 25% |
| Yastimadhu | Glycerrhiza glabra | 12.5% |
| Haridra | Curcuma longa | 12.5% |
| Sunthi | Zingiber officinale | 2% |
| Vasaka | Adhatoda vasika | 16.66% |
| Kulanjan | Alpinia galanga | 5.00% |
| Brahati | Solanum indicum | 4.32% |
| Pippali | Piper longum | 5.00% |
| Vibhitak | Terminalia belerica | 6.6% |
| Ghritakumari | Aloe barbadensis | 10.4% |

The said composition is so adjusted as to deliver 400 mg of the total extracts in 10 ml of the liquid formulation.

### Example 6

The composition employed was similar to that in Example 5, except that the spray dried plant extracts used are semisolid in nature i.e. soft extract.

### Example 7

The product has been formulated in the liquid dosage form using similar manufacturing procedure as mentioned in example 1 and having the following composition of active ingredients:

Each 10 ml contains extracts derived from:

| Name of Plant | Botanical name | Qty in % |
|---|---|---|
| Tulsi | Ocimum sanctum | 25% |
| Yastimadhu | Glycerrhiza glabra | 15.4% |
| Haridra | Curcuma longa | 12.82% |
| Sunthi | Zingiber officinale | 2.56% |
| Vasaka | Adhatoda vasika | 15.42% |
| Brahati | Solanum indicum | 5.13% |
| Kushta | Saussurea lappa | 2.56% |
| Sugadha muricha | Piper cubeba | 2.56% |
| Vibhitak | Terminalia belerica | 5.13% |
| Ghritakumari | Aloe barbadensis | 12.82% |

The said composition is so adjusted as to deliver 400 mg of the total extracts in 10 ml of the liquid formulation.

### Example 8

The composition employed was similar to that in Example 7, except that the spray dried plant extracts used are semisolid in nature i.e. soft extract.

### Example 9

The product has been formulated in the liquid dosage form using similar manufacturing procedure as mentioned in example 1 and having the following composition of active ingredients:

Each 10 ml contains extracts derived from:

| Name of the Ingredient | | Quantity in % |
|---|---|---|
| Tulsi | Ocimum sanctum | 14.3% |
| Yastimadhu | Glycyrrhiza glabra | 8.47% |
| Haridra | Curcuma longa | 7.53% |
| Sunthi | Zingiber officinale | 1.88% |
| Vasaka | Adhatoda vasica | 7.53% |
| Pushkarmool | Inula racemosa | 1.88% |
| Ghritkumari | Aloe barbadensis | 4.7% |
| Kantakari | Solanum xanthocarpum | 11.3% |
| Pippali | Piper longum | 2.35% |
| Haritaki | Terminalia chebula | 7.53% |
| Amalki | Emblica officinalis | 8.47% |
| Bharangi | Clerodendron serratum | 23.5% |

The said composition is so adjusted as to deliver 400 mg of the total extracts in 10 ml of the liquid formulation.

### Example 10

The composition employed was similar to that in Example 9, except that the spray dried plant extracts used are semisolid in nature i.e. soft extract.

It is to be understood that the aforesaid examples and embodiments described herein above are for the purposes of providing a description of the present invention by way of examples and not to be viewed as limiting the preset invention in any way. Various modifications or changes that may be made to that described herein above by those of ordinary skill in the art are also contemplated by the present invention and are to be included within the spirit and purview of this application and the following claims.

Dosage:

The effective doses of Herbal liquid Syrup was found to be as follows:

Curative dose for adults: 1-2 teaspoons thrice daily, orally with cold water or as directed by the physician.

Curative dose for: ½-1 teaspoons thrice children daily, orally with cold water or as directed by the physician.

The dose administered and treatment regiment will be dependant for example on the diseases, the severity thereof on the patient being treated and his response to treatment and therefore may be widely varied.

We claim:

1. Oral formulations in the form of a herbal liquid syrup which
a. contains therapeutically active herbal ingredients providing relief in the respiratory tract diseases viz. cough, cold, allergic asthma, seasonal allergic rhinitis, pharyngitis, laryngitis, whooping cough and the like
b. which are non sedative, non-alcoholic, nonfreezing and
c. is characterized in that the herbal ingredients comprise extracts derived from Ayurvedic plants selected from the following group:

| Name of Plant | Botanical name | Qty in % |
|---|---|---|
| Tulsi | Ocimum sanctum | 10-40% |
| Yastimadhu | Glycerrhiza glabra | 10-25% |
| Haridra | Curcuma longa | 2-20% |
| Sunthi | Zingiber officinale | 1-10% |
| Vasaka | Adhatoda vasika | 10-25% |
| Brahati | Solanum indicum | 1-10% |
| Kushta | Saussurea lappa | 1-5% |
| Sugadha muricha | Piper cubeba | 1-5% |
| Vibhitak | Terminalia belerica | 1-10% |
| Ghritakumari | Aloe barbadensis | 10-30% |
| Pushkarmool | Inula racemosa | 2-10% |
| Bhurangi | Clerodendrum serratum | 1-5% |
| Kantakari | Solanum xanthocorpum | 1-5% |
| Pipalli | Pipper longum | 1-10% |
| Kulanjan | Alpinia galanga | 1-5% |
| Hartaki | Terminalia chebula | 5-15% |
| Amalki | Embelica officinalis | 5-15% |

d. The process for preparing the herbal composition of the present invention as stated in claim 1 comprises procuring, cleaning, grading of specified herbal plants as per the standard specification, disintegrating and pulverizing separately to form the coarse powder, then macerating individual powders separately in aqueous solution of preservatives and then extracting, filtering and concentrating and spray drying to make dry extract powder or a semisolid paste. Further dissolving each of the extracts separately in aqueous solution containing preservatives and then allowing to maturate after mixing. Then adding filtered extracts to the sugar syrup containing preservatives, glycerin and appropriate excipients to obtain a homogenous cough syrup.

2. Formulations as stated in claim 1, characterized in that each 10 ml thereof comprises extracts derived from the following material:

| Name of Plant | Botanical name | Qty in % |
|---|---|---|
| Tulsi | Ocimum sanctum | 25% |
| Yastimadhu | Glycerrhiza glabra | 15% |
| Haridra | Curcuma longa | 12.5% |
| Sunthi | Zingiber officinale | 2.5% |
| Vasaka | Adhatoda vasika | 15% |
| Brahati | Solanum indicum | 5% |
| Sugadha muricha | Piper cubeba | 2.5% |
| Vibhitak | Terminalia belerica | 5% |
| Ghritakumari | Aloe barbadensis | 12.5% |
| Pushkarmool | Inula racemosa | 5% |

3. Formulations as stated in claim 1, characterized in that each 10 ml thereof comprises extracts derived from the following material:

| Name of Plant | Botanical name | Qty in % |
|---|---|---|
| Tulsi | Ocimum sanctum | 21.27% |
| Yastimadhu | Glycerrhiza glabra | 12.67% |
| Haridra | Curcuma longa | 12.67% |
| Sunthi | Zingiber officinale | 3.19% |
| Vasaka | Adhatoda vasika | 17% |
| Vibhitak | Terminalia belerica | 5.32% |
| Ghritakumari | Aloe barbadensis | 10.64% |
| Bhurangi | Clerodendrum serratum | 8.5% |
| Kantakari | Solanum xanthocorpum | 4.25% |
| Pipalli | Pipper longum | 4.25% |

4. Formulations as stated in claim 1, characterized in that each 10 ml thereof comprises extracts derived from the following material:

| Name of Plant | Botanical name | Qty in % |
|---|---|---|
| Tulsi | Ocimum sanctum | 25% |
| Yastimadhu | Glycerrhiza glabra | 12.5% |
| Haridra | Curcuma longa | 12.5% |
| Sunthi | Zingiber officinale | 2% |
| Vasaka | Adhatoda vasika | 16.66% |
| Brahati | Solanum indicum | 4.32% |
| Vibhitak | Terminalia belerica | 6.6% |
| Ghritakumari | Aloe barbadensis | 10.4% |
| Pipalli | Pipper longum | 5.00% |
| Kulanjan | Alpinia galanga | 5.00% |

5. Formulations as stated in claim 1, characterized in that each 10 ml thereof comprises extracts derived from the following material:

| Name of Plant | Botanical name | Qty in % |
|---|---|---|
| Tulsi | Ocimum sanctum | 25.6% |
| Yastimadhu | Glycerrhiza glabra | 15.4% |
| Haridra | Curcuma longa | 12.82% |
| Sunthi | Zingiber officinale | 2.56% |
| Vasaka | Adhatoda vasika | 15.42% |
| Brahati | Solanum indicum | 5.13% |
| Kushta | Saussurea lappa | 2.56% |
| Sugadha muricha | Piper cubeba | 2.56% |
| Vibhitak | Terminalia belerica | 5.13% |
| Ghritakumari | Aloe barbadensis | 12.82% |

6. Formulations as stated in claim 1, characterized in that each 10 ml thereof comprises extracts derived from the following material:

| Name of the Ingredient | | Quantity per 100 ml |
|---|---|---|
| Tulsi | Ocimum sanctum | 14.3% |
| Yastimadhu | Glycyrrhiza glabra | 8.47% |
| Haridra | Curcuma longa | 7.53% |
| Sunthi | Zingiber officinale | 1.88% |
| Vasaka | Adhatoda vasica | 7.53% |
| Pushkarmool | Inula racemosa | 1.88% |
| Ghritkumari | Aloe barbadensis | 4.7% |
| Kantakari | Solanum xanthocarpum | 11.3% |
| Pippali | Piper longum | 2.35% |
| Haritaki | Terminalia chebula | 7.53% |
| Amalki | Emblica officinalis | 8.47% |
| Bharangi | Clerodendron serratum | 23.5% |

7. A process for the manufacture of formulations as stated in claim 1, comprising the following steps:
i) Procuring of following herbal plants as per the standard specification. For the purpose of this invention the Ayurvedic plants constituents are selected from the following group:

| Botanical name | | |
|---|---|---|
| of Plant | Plant constituents | Qty. in % |
| Ocimum sanctum | leaves, seeds and root. | 10-40% |
| Glycerrhiza glabra | peeled root, | 10-25% |
| Zingiber officinale | scraped and dried rhizomes as | 1-10% |
| | well as the green ones. | |
| Gurcuma longa | Tubers and rhizomes. | 2-20% |
| Adhatoda vasika | Leaves, roots, flowers and bark. | 10-25% |
| Solanum indicum | Roots, fruits and seeds. | 1-10% |
| Inula racemosa | Roots | 2-10% |
| Piper cubeba | Dried immature full grown fruits | 1-5% |
| | called the cubebs. | |
| Terminalia belerica | Fruits. | 1-10% |
| Aloe barbadensis | Expressed and dried juice | 10-30% |
| | of leaves and pulp. | |
| Saussurca Lappa | Roots. | 1-5% |
| Clerodendron | Roots, Leaves. | 1-5% |
| serratum | | |
| Solanum | Roots, stem, leaves, flowers | 1-5% |
| xanthocorpum | fruits (panchang). | |
| Piper longum | Immature berries(i.e.dried, 1-5% unripe | 1-5% |
| | fruits or fruiting spikes, dried in the | |
| | sun and roots. | |
| Alpinia galanga | Rhizome, fruit | 1-5% |
| Terminalia chebula | Fruits | 5-15% |
| Embelica officinalis | Fresh and dried fruits | 5-15% |

ii). Disintegrating the said herbal plants as given in Step (i) separately in the disintegrater in a conventional manner and pulverizing separately the same in the pulveriser to form the—coarse powder,
iii). Macerating the above herbs of step (1) separately by soaking the same in aqueous solution of preservatives such as Sodium benzoate Sodium methylparaben, Sodium propylparaben, Sorbic acid, extracting with stirring for 18 hours at 70-80° C. which is filtered, concentrated under vacuum and spray dried to obtain semisolid soft extract or dry powder extracts,
iv) Dissolving separately the extracts of step (iii) in aqueous solution containing requisite quantity of preservatives, mixing together, the resulting solution is kept for maturation at 20-25° C. for 18 hrs and filtered,
v) Preparing sugar solution by dissolving in aqueous solution containing appropriate quantity of sugar, citric acid, glycerin, preservatives to obtain clear homogenous syrup and adding it to step (iv).
v) Adding colours, menthol and other flavouring agents and the commonly known pharmaceutical excipients as required, to make this syrup palatable for patients' acceptability.
